(19) United States
(12) Patent Application Publication
(10) Pub. No.: US 2023/0264223 A1
(43) Pub. Date: Aug. 24, 2023

(54) FAST GLUE REPLENISHMENT DEVICE AND METHOD BY USING DISPENSING CARTRIDGE

(71) Applicant: LIAO-CHUAN HUANG, TAOYUAN CITY (TW)

(72) Inventor: LIAO-CHUAN HUANG, TAOYUAN CITY (TW)

(21) Appl. No.: 18/078,721

(22) Filed: Dec. 9, 2022

(30) Foreign Application Priority Data
Feb. 23, 2022 (TW) 111106596

(51) Int. Cl. B05C 5/02; G05D 16/20

(52) U.S. Cl. CPC B05C 5/0225; G05D 16/2013

(57) ABSTRACT

A fast glue replenishment device by using a dispensing cartridge is illustrated, which has a power unit, a regulation unit, a gas container, a control unit and a dispensing cartridge. The regulation unit controls a gas pressure of a gas output by the power unit to be equal to a necessary gas pressure value which the dispensing cartridge pushes a supply material for dispensing. When the dispensing cartridge pushes the supply material for dispensing, the gas container continuously provides the gas having the gas pressure value into the dispensing cartridge by using the control unit, such that the dispensing cartridge is maintained to have the necessary gas pressure value which the dispensing cartridge pushes the supply material for dispensing. Thus, the supply material is pushed in a short path and in a time differential to increase accuracy of pushing the supply material for dispensing.

### TECHNICAL FIELD

The present disclosure relates to a fast glue replenishment device and method by using a dispensing cartridge, and in particular to, a fast glue replenishment device and method by using a dispensing cartridge, both of which can push a supply material in a short path and in a time differential to increase accuracy of pushing the supply material for dispensing.

### RELATED ART

Along with the progress of science and technology, quality requirements of the electronic parts of precision and complex structure in electronic industry, medical equipment and optics fields become more and more higher, therefore, even if a process misses in the manufacturing process, it will lead to poor product quality. It is noted that, in the manufacturing process, a dispensing process is also one of the important processes.

A conventional dispensing manner adopts a manual mode to operate, but the speed of manual operation is slow and precision is low, which cannot satisfy present demand far away. Therefore, automatic dispensers have appeared on the market, and a large number of dispensing operations are carried out quickly through automated dispensing technology. However, at present, the dispenser that uses pneumatic control to dispense still has the problem of uneven air pressure control during operations. When the air pressure of the dispensing cartridge of the dispenser drops, it is easy to cause the dispensing amount to be inconsistent, thereby affecting the quality of the product.

When the conventional dispensing cartridge pushes a supply material for dispensing, the air pressure drops to a certain level after a period of operation. Then, the gas pressure value is manually increased to a specific value for the normal operation to carry out subsequent dispensing operations. However, this manner is also difficult to ensure that the amount of glue dispensed is consistent each time.

Further, the prior art discloses a pressure compensation system for a liquid level variation, which can refer to the invention patent No. 1658873 of the Republic of China. The conventional pressure compensation system for the liquid level variation comprises a container, a pressure supply, a non-contact liquid level sensor and a processor. An accommodating space of the container accommodates a liquid. The pressure supply provides a pressure to the accommodating space, such that the liquid is output from an outlet of the container. The non-contact liquid level sensor is disposed in the accommodating space and above the liquid surface, and configured to detect a liquid level variation of the liquid to generate a variation signal. The processor has a control module, and the control module controls the pressure supply according to the variation signal. The pressure supply can regulate the pressure, to make the liquid output amount be consistent. Thus, it achieves consistent precision of automatic liquid output control.

Refer to the dotted curve line in FIG. 6, when the conventional dispensing cartridge pushes the enzyme on the blood glucose test strip for dispensing, the non-contact liquid level sensor detects the liquid level variation of the liquid to generate the variation signal, and the control module then regulates the pressure of the pressure supply according to the variation signal. Since the whole signal propagation path is longer, it results that the response time for regulating the pressure is also longer. However, regarding the fast and automatic dispensing manner, the gas pressure value is hard to be restored to the specific value for the normal operation, and it affects the control of the inconsistent amount of pushing supply material at the same time, which causes the problem of uneven product quality.

### SUMMARY

Accordingly, the present disclosure provides a fast glue replenishment device and method by using a dispensing cartridge to solve the problem of inconsistent dispensing amount caused by the use of conventional dispensers.

An objective of the present disclosure is to provide a fast glue replenishment device and method by using a dispensing cartridge, both of which utilizes a regulation unit (such as, a pressure regulator valve) to control a gas pressure value in the gas container, and then a control unit (such as, an electromagnetic valve) turns on or off to continuously control the gas to enter dispensing cartridge. Thus, a supply material is pushed in a short path and in a time differential to increase accuracy of pushing the supply material for dispensing.

To solve the above problem, a fast glue replenishment device by using a dispensing cartridge comprises: a power unit, having an air outlet, configured to provide a gas; a regulation unit, communicative to the air outlet of the power unit, configured to control the power unit to output the gas having a gas pressure value; a gas container, communicative to the regulation unit, configured to store the gas having the gas pressure value; a control unit, communicative to the gas container, having a switch; and the dispensing cartridge, having an accommodating space communicative to an air inlet and a feed port, wherein the air inlet is communicative to the control unit; wherein the regulation unit regulates the gas pressure value of the gas output from the power unit to be equal to a necessary gas pressure value which the dispensing cartridge pushes a supply material for dispensing, when the dispensing cartridge pushes the supply material for dispensing, the gas container continuously provides the gas having the gas pressure value into the dispensing cartridge by using the control unit, such that the dispensing cartridge is maintained to have the necessary gas pressure value which the dispensing cartridge pushes the supply material for dispensing.

In one embodiment, the power unit is a gas pressure source.

In one embodiment, the regulation unit is a pressure regulator valve.

In one embodiment, the control unit is an electromagnetic valve.

In one embodiment, the control unit utilizes the switch to control the gas in the gas container enters the dispensing cartridge or not.

In one embodiment, the control unit further comprises a timing controller, and the timing controller controls an on/off period and/or on/off times of the switch.

In one embodiment, the dispensing cartridge further comprises a gas pressure monitor, and the gas pressure monitor monitors the gas pressure value of the gas in the accommodating space.

In one embodiment, the dispensing cartridge further comprises a push rubber piston part, and the push rubber piston part is slidably disposed in the accommodating space.

To solve the above problem, a fast glue replenishment method by using a dispensing cartridge comprises: a step of outputting a gas; a step of regulating the gas to have a gas pressure value; a step of storing the gas having the gas pressure value; a step of controlling the gas having the gas pressure value to be output; and a step of outputting the gas having the gas pressure value into a dispensing cartridge; wherein the gas pressure of the gas is regulated to be equal to a necessary gas pressure value which the dispensing cartridge pushes a supply material for dispensing, when the dispensing cartridge pushes the supply material for dispensing, the gas having the gas pressure value is continuously provided into the dispensing cartridge, such that the dispensing cartridge is maintained to the necessary gas pressure value which the dispensing cartridge pushes the supply material for dispensing.

In one embodiment, in the step of outputting the gas, the gas is provided by a power unit.

In one embodiment, in the step of regulating the gas to have the gas pressure value, the gas is regulated to have the gas pressure value by a regulation unit.

In one embodiment, in the step of storing the gas having the gas pressure value, the gas having the gas pressure value is stored in a gas container.

In one embodiment, in the step of controlling the gas having the gas pressure value to be output, the gas having the gas pressure value is controlled to be output by a switch of a control unit.

In one embodiment, the control unit utilizes the switch to control the gas in the gas container enters the dispensing cartridge or not.

In one embodiment, the fast glue replenishment method by using the dispensing cartridge further comprises a step of timing controlling, and in the step of timing controlling, an on/off period and/or on/off times of the switch is controlled by a timing controller.

In one embodiment, in the step of outputting the gas having the gas pressure value into the dispensing cartridge, the dispensing cartridge has an accommodating space communicative to an air inlet and a feed port, and the air inlet is communicative to the control unit.

In one embodiment, the fast glue replenishment method by using the dispensing cartridge further comprising a step of monitoring the gas pressure, wherein in the step of monitoring the gas pressure, the gas pressure of the gas in the accommodating space is monitored by a gas pressure monitor

In one embodiment, the dispensing cartridge further comprises a push rubber piston part, and the push rubber piston part is slidably disposed in the accommodating space.

Above-mentioned purposes and advantages of the present disclosure can be understood and known from the detailed descriptions and accompanying drawings of following exemplary embodiments. The present disclosure will be described in detail below with the following examples and drawings.

### BRIEF DESCRIPTIONS OF DRAWINGS

FIG. 1 is a schematic structure block diagram of a fast glue replenishment device by using a dispensing cartridge according to one embodiment of the present disclosure.

FIG. 2 is a schematic structure diagram of a fast glue replenishment device by using a dispensing cartridge according to one embodiment of the present disclosure.

FIG. 3 is a schematic enlarged diagram of a fast glue replenishment device by using a dispensing cartridge according to one embodiment of the present disclosure.

FIG. 4 is a schematic pressure supply block diagram of a fast glue replenishment device by using a dispensing cartridge according to one embodiment of the present disclosure.

FIG. 5 is a schematic flowchart of a fast glue replenishment method by using a dispensing cartridge according to one embodiment of the present disclosure.

FIG. 6 is a schematic comparison diagram of the fast glue replenishment device by using the dispensing cartridge of the present disclosure and conventional glue replenishment device when both of them are applied to the blood glucose test strip.

### DETAILS OF EXEMPLARY EMBODIMENTS

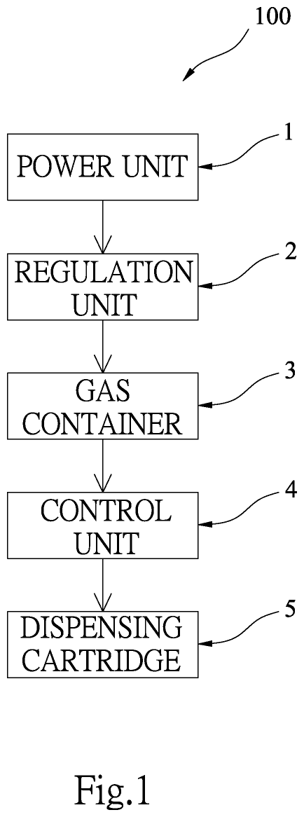

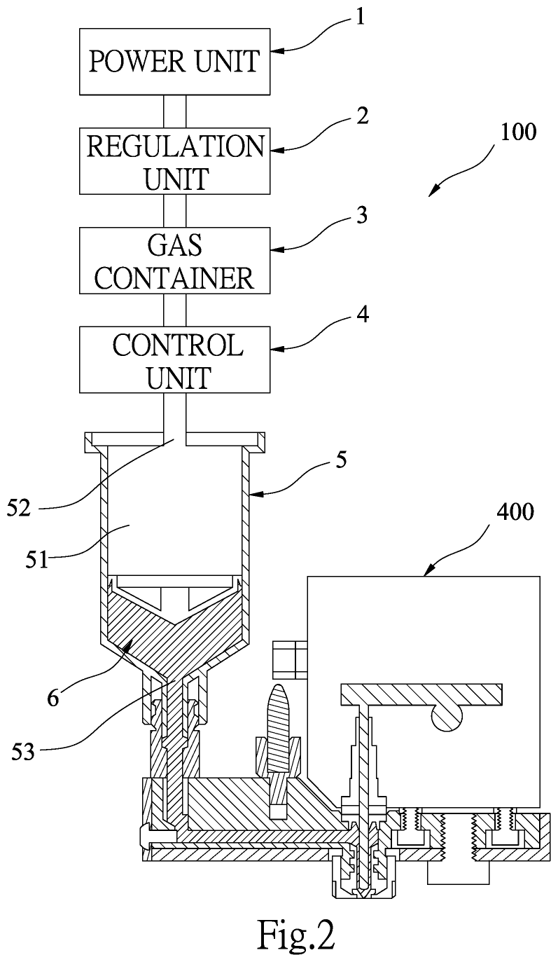

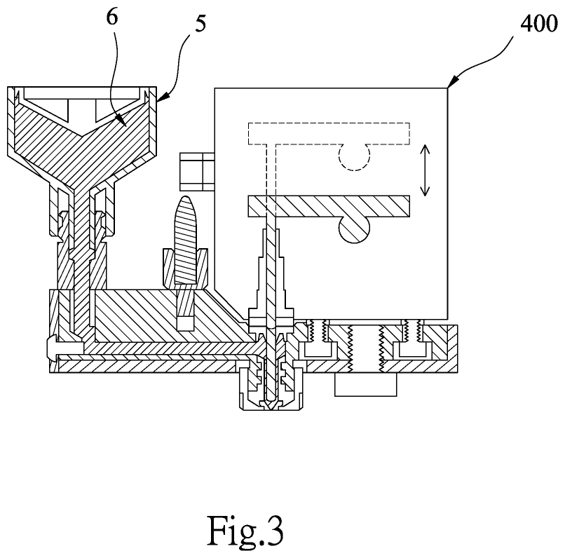

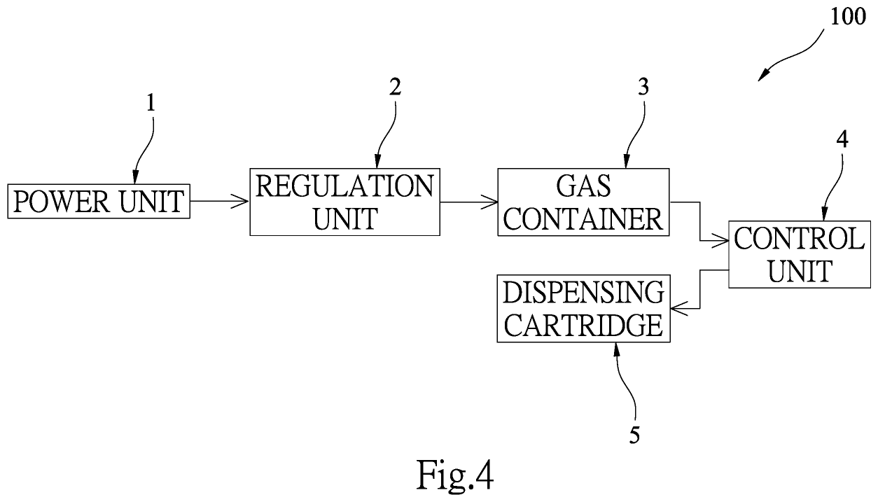

Refer to FIG. 1 to FIG. 4, FIG. 1 is a schematic structure block diagram of a fast glue replenishment device by using a dispensing cartridge according to one embodiment of the present disclosure, FIG. 2 is a schematic structure diagram of a fast glue replenishment device by using a dispensing cartridge according to one embodiment of the present disclosure, FIG. 3 is a schematic enlarged diagram of a fast glue replenishment device by using a dispensing cartridge according to one embodiment of the present disclosure, and FIG. 4 is a schematic pressure supply block diagram of a fast glue replenishment device by using a dispensing cartridge according to one embodiment of the present disclosure.

As shown in FIG. 1 to FIG. 4, the fast glue replenishment device (100) by using a dispensing cartridge (5) comprises a power unit (1), a regulation unit (2), a gas container (3), a control unit (4) and the dispensing cartridge (5). The fast glue replenishment device (100) by using the dispensing cartridge (5) can be used in a dispenser (400). The fast glue replenishment device (100) by using dispensing cartridge (100) can perform dispensing or glue replenishment, and the glue can be silicone gel, silicone oil, UV glue, solder paste or conductive adhesive.

The dispenser (400) at least comprises a dispensing cartridge supporting structure and a displacement adjusting structure. The dispensing cartridge supporting structure is configured to support the fast glue replenishment device (100) by using the dispensing cartridge (5). The dispensing cartridge supporting structure can also comprise an angle adjusting unit, and the angle adjusting unit is configured to a tilted angle which the fast glue replenishment device (100) by using the dispensing cartridge (5) dispenses. The displacement adjusting structure has a first driving device slidably disposed in an X-axis rail, a second driving device slidably disposed in a Y-axis rail and a third driving device slidably disposed in a Z-axis rail. The displacement adjusting structure can drive the dispensing cartridge supporting structure to displace in an X-axis direction, a Y-axis direction or a Z-axis direction.

The power unit (1) is configured to provide a gas, which acts as a power source of the fast glue replenishment device (100) by using the dispensing cartridge (5) when pushing the supply material (6) for dispensing. The power unit (1) has an air outlet, and the air outlet can transfer the gas to the gas container (3). In some embodiments, the power unit (1) can be a gas pressure source, for example, an air compressor, and the present disclosure is not limited thereto.

The regulation unit (2) is communicative to the air outlet of the power unit (1). The regulation unit (2) controls the power unit (1) to output the gas having a gas pressure value. The regulation unit (2) can regulates the gas pressure value of the gas according to actual requirements. In some embodiments, the regulation unit (2) can be a pressure regulator valve and communicative to the power unit (1) of the gas pressure source.

The gas container (3) is communicative to the regulation unit (2). The gas container (3) stores the gas having the gas pressure value. In some embodiments, the gas container (3) can be a gas storage bottle, and can be communicative to the regulation unit (2) of the pressure regulator valve. In practice, the gas container (3) pre-stores the gas having the gas pressure value, so as to fast refill the gas required when fast pushing the supply material (6) for dispensing.

The control unit (4) is communicative to the gas container (3). The control unit (4) has a switch. The control unit (4) utilizes the switch to control gas in the gas container (3) to enter the dispensing cartridge (5) or not. In some embodiments, the control unit (4) is an electromagnetic valve. In practice, an on/off period and on/times of the switch is controlled according to the dispensing amount and dispensing time of pushing supply material for dispensing each time, so as to precisely refill the gas required when pushing the supply material (6) for dispensing.

The dispensing cartridge (5) has an accommodating space (51). The accommodating space (51) is communicative to an air inlet (52) and a feed port (53). The air inlet (52) is communicative to the control unit (4). The accommodating space (51) accommodates the supply material (6) (for example, gel fluid). The control unit (4) controls the gas in the gas container (3) to enter the accommodating space (51) of the dispensing cartridge (5) via the air inlet (52), so as to refill the gas required when pushing the supply material (6) for dispensing. The regulation unit (2) regulates the gas pressure value of the gas output from the power unit (1) to be equal to a necessary gas pressure value which the dispensing cartridge (5) pushes a supply material (6) for dispensing. When the dispensing cartridge (5) pushes the supply material (6) for dispensing, the gas container (3) continuously provides the gas having the gas pressure value into the dispensing cartridge (5) by using the control unit (4), such that the dispensing cartridge (5) is maintained to have the necessary gas pressure value which the dispensing cartridge (5) pushes the supply material (6) for dispensing.

In practice, the fast glue replenishment device (100) by using dispensing cartridge (5) can be installed in the dispenser (400). When the dispenser (400) pushes the supply material (6) for dispensing, the gas container (3) provides the gas required by the dispensing cartridge (5) under the control of the control unit (4). The regulation unit (2) controls the gas pressure value of the gas output by the power unit (1) to be equal to the necessary gas pressure value which the dispensing cartridge (5) pushes a supply material (6) for dispensing. Therefore, when the dispenser (400) fast automatically dispenses, the gas having the necessary gas pressure value which the dispensing cartridge (5) pushes a supply material (6) for dispensing can be continuously refilled, the content of the supply material (6) for dispensing each time can be maintained, which maintains a good yielding efficiency and a good production quality.

In one embodiment of the present disclosure, the control unit (4) further comprises a timing controller, and the timing controller controls an on/off period and/or on/off times of the switch. When the dispenser (400) fast automatically dispenses, the time controller is directly utilized to refill the gas during dispensing process. Thus, extra installation of sensors for sensing the gas pressure can be reduced, the response time for refilling the gas after sensing the gas pressure can be also reduced, and it avoids the inconsistent dispensing amount of the pushed supply material (6) caused by the longer response time of the sensors.

In one embodiment of the present disclosure, the dispensing cartridge (5) further comprises a gas pressure monitor. The gas pressure monitor monitors the gas pressure value of the gas in the accommodating space (51). When the dispenser (400) pushes the supply material (6), the user can utilize the gas pressure monitor to continuously monitor whether the gas pressure value is maintained to be consistent or the normal gas pressure value when normally pushing the supply material (6).

In one embodiment of the present disclosure, the dispensing cartridge (5) further comprises a push rubber piston part, and the push rubber piston part is slidably disposed in the accommodating space (51). When the control unit (4) controls the gas having the gas pressure value to be output into the dispensing cartridge (5), the push rubber piston part is used push the supply material (6) in the accommodating space (51) to be output to the feed port (53).

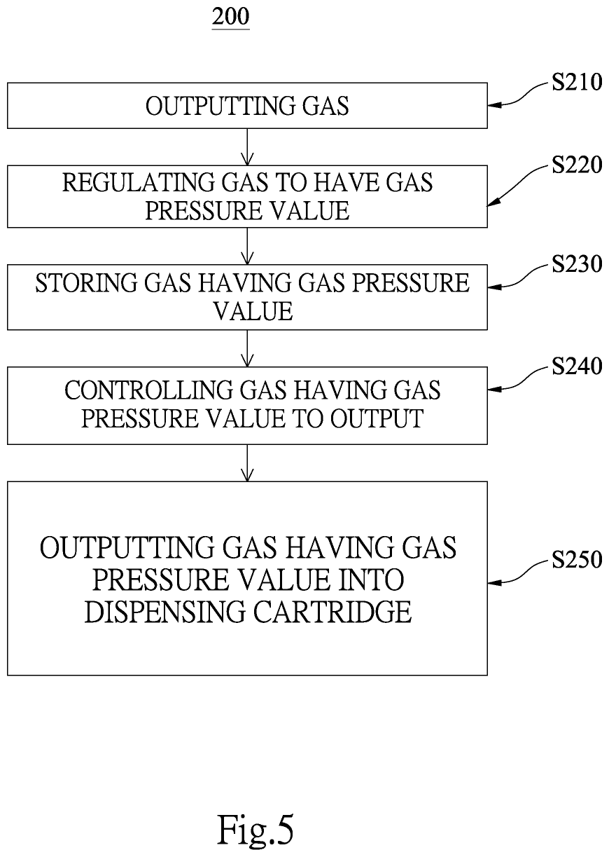

Refer to FIG. 1 to FIG. 5, and FIG. 5 is a schematic flowchart of a fast glue replenishment method by using a dispensing cartridge according to one embodiment of the present disclosure. The present disclosure provides a fast glue replenishment method (200) by using the dispensing cartridge (5) comprises steps as follows. In a step (S210) of outputting the gas, the gas is provided by the power unit (1). In a step (S220) of regulating the gas to have the gas pressure value, the gas is regulated to have the gas pressure value by the regulation unit (2). In a step (S230) of storing the gas having the gas pressure value, the gas having the gas pressure value is stored in the gas container (3). In a step (S240) of controlling the gas having the gas pressure value to be output, the gas having the gas pressure value is controlled to be output by a switch of a control unit, and specifically, the control unit (4) utilizes the switch to control the gas in the gas container (3) to enter the dispensing cartridge (5) or not. In a step (S250) of outputting the gas having the gas pressure value into the dispensing cartridge (5), the dispensing cartridge (5) has the accommodating space (51) communicative to the air inlet (52) and the feed port (53), and the air inlet (52) is communicative to the control unit (4), wherein the gas pressure value of the gas output from the power unit (1) is regulated to be equal to the necessary gas pressure value which the dispensing cartridge (5) pushes a supply material (6) for dispensing. When the dispensing cartridge (5) pushes the supply material (6) for dispensing, the gas container (3) continuously provides the gas having the gas pressure value into the dispensing cartridge (5) by using the control unit (4), such that the dispensing cartridge (5) is maintained to have the necessary gas pressure value which the dispensing cartridge (5) pushes the supply material (6) for dispensing. The details and implementations of the fast glue replenishment method (200) by using the dispensing cartridge (5) can be the same as the above mentioned component details of the fast glue replenishment device (100) by using the dispensing cartridge (5), and the redundant descriptions are thus omitted.

In one embodiment of the present disclosure, the fast glue replenishment method (200) by using the dispensing cartridge (5) further comprises a step of timing controlling, and in the step of timing controlling, an on/off period and/or on/off times of the switch is controlled by a timing controller. When the dispenser (400) fast automatically dispenses, the time controller is directly utilized to refill the gas during dispensing process. Thus, extra installation of sensors for sensing the gas pressure can be reduced, the response time for refilling the gas after sensing the gas pressure can be also reduced, and it avoids the inconsistent dispensing amount of the pushed supply material (6) caused by the longer response time of the sensors.

In one embodiment of the present disclosure, the fast glue replenishment method (200) by using the dispensing cartridge (5) further comprises a step of monitoring the gas pressure, in the step of monitoring the gas pressure, the gas pressure of the gas in the accommodating space (51) is monitored by a gas pressure monitor. When the dispenser (400) pushes the supply material (6), the user can utilize the gas pressure monitor to continuously monitor whether the gas pressure value is maintained to be consistent or the normal gas pressure value when normally pushing the supply material (6).

In one embodiment of the present disclosure, the dispensing cartridge (5) used in the fast glue replenishment method (200) further comprises a push rubber piston part, and the push rubber piston part is slidably disposed in the accommodating space (51). When the control unit (4) controls the gas having the gas pressure value to be output into the dispensing cartridge (5), the push rubber piston part is used push the supply material (6) in the accommodating space (51) to be output to the feed port (53).

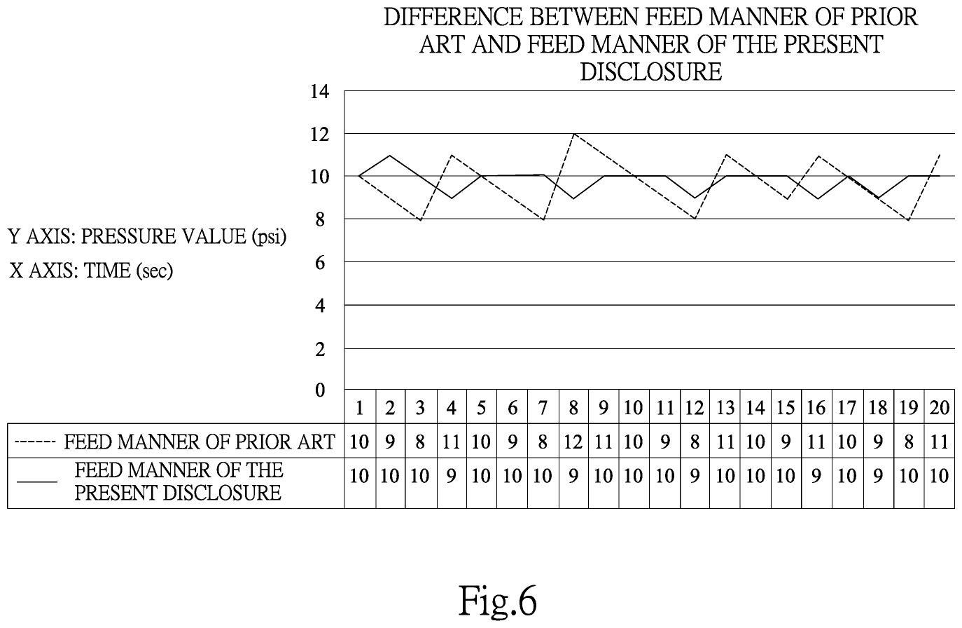

Refer to FIG. 1 to FIG. 6, and FIG. 6 is a schematic comparison diagram of the fast glue replenishment device by using the dispensing cartridge of the present disclosure and conventional glue replenishment device when both of them are applied to the blood glucose test strip. Under normal circumstances, the air pressure source obtained from the equipment on the production line is about 98 psi, and the pressure value (air pressure) for pushing the supply material (6) via the air pressure is about 3 psi to 56 psi (depending on the fluid viscosity). The stability of fluid stacking is directly related to air pressure, such as pressure value accuracy and compensation time difference and viscosity, so how to maintain the necessary pressure value for fast (small time difference) and accurate (pressure accuracy) for pushing the supply material (6) is the focus of the control. The dispenser (400) pushes the supply material (6) of enzyme to dispense on the blood glucose test strip, and the pressure curve of the supply material (6) showing its values in time is obtained as shown in FIG. 6. In the test, it shows the gas pressure value variation in the dispensing cartridge (5) each second after continuously dispensing 20 seconds. The gas output by the power unit (1) has the pressure value of 98 psi, by using the fast glue replenishment device (100) or method (200) by using the dispensing cartridge (5) of the present disclosure to push supply material (6) of enzyme on the blood glucose test strip, the necessary gas pressure value of the supply material (6) when the dispensing cartridge (5) push the supply material (6) for dispensing is about 9 psi to 10 psi. However, by using the prior art, the gas pressure value in the dispensing cartridge is about 8 psi to 12 psi. Thus, compared with the prior art, the dispensing cartridge (5) of the present disclosure can better maintain the necessary gas pressure value required of pushing the supply material (6) for dispensing, so as to reduce the excessive variation of gas pressure value of the prior art which affects the amount of supply material during dispensing.

To sum up, the present disclosure utilizes the regulation unit of the pressure regulator valve to control the gas having the gas pressure value to be output, and then pre-stores the gas having the gas pressure value in the gas container. When the dispenser fast automatically dispenses, the control unit of the electromagnetic valve turns on or off to continuously control the gas to enter dispensing cartridge. Thus, the necessary gas pressure value which the dispensing cartridge push the supply material for dispensing can be maintained since the supply material is pushed in a short path and in a time differential, and the accuracy of pushing the supply material for dispensing is increased.

The above-mentioned descriptions represent merely the exemplary embodiment of the present disclosure, without any intention to limit the scope of the present disclosure thereto. Various equivalent changes, alternations or modifications based on the claims of present disclosure are all consequently viewed as being embraced by the scope of the present disclosure.

What is claimed is:

1. A fast glue replenishment device by using a dispensing cartridge, comprising:
a power unit, having an air outlet, configured to provide a gas;
a regulation unit, communicative to the air outlet of the power unit, configured to control the power unit to output the gas having a gas pressure value;
a gas container, communicative to the regulation unit, configured to store the gas having the gas pressure value;
a control unit, communicative to the gas container, having a switch; and
the dispensing cartridge, having an accommodating space communicative to an air inlet and a feed port, wherein the air inlet is communicative to the control unit;
wherein the regulation unit regulates the gas pressure value of the gas output from the power unit to be equal to a necessary gas pressure value which the dispensing cartridge pushes a supply material for dispensing, when the dispensing cartridge pushes the supply material for dispensing, the gas container continuously provides the gas having the gas pressure value into the dispensing cartridge by using the control unit, such that the dispensing cartridge is maintained to have the necessary gas pressure value which the dispensing cartridge pushes the supply material for dispensing.

2. The fast glue replenishment device by using the dispensing cartridge of claim 1, wherein the power unit is a gas pressure source.

3. The fast glue replenishment device by using the dispensing cartridge of claim 1, wherein the regulation unit is a pressure regulator valve.

4. The fast glue replenishment device by using the dispensing cartridge of claim 1, wherein the control unit is an electromagnetic valve.

5. The fast glue replenishment device by using the dispensing cartridge of claim 1, wherein the control unit utilizes the switch to control the gas in the gas container enters the dispensing cartridge or not.

6. The fast glue replenishment device by using the dispensing cartridge of claim 1, wherein the control unit further comprises a timing controller, and the timing controller controls an on/off period and/or on/off times of the switch.

7. The fast glue replenishment device by using the dispensing cartridge of claim 1, wherein the dispensing cartridge further comprises a gas pressure monitor, and the gas pressure monitor monitors the gas pressure value of the gas in the accommodating space.

8. The fast glue replenishment device by using the dispensing cartridge of claim 1, wherein the dispensing cartridge further comprises a push rubber piston part, and the push rubber piston part is slidably disposed in the accommodating space.

9. A fast glue replenishment method by using a dispensing cartridge, comprising:
a step of outputting a gas;
a step of regulating the gas to have a gas pressure value;
a step of storing the gas having the gas pressure value;
a step of controlling the gas having the gas pressure value to be output; and
a step of outputting the gas having the gas pressure value into the dispensing cartridge;
wherein the gas pressure of the gas is regulated to be equal to a necessary gas pressure value which the dispensing cartridge pushes a supply material for dispensing, when the dispensing cartridge pushes the supply material for dispensing, the gas having the gas pressure value is continuously provided into the dispensing cartridge, such that the dispensing cartridge is maintained to the necessary gas pressure value which the dispensing cartridge pushes the supply material for dispensing.

10. The fast glue replenishment method by using the dispensing cartridge of claim 9, wherein in the step of outputting the gas, the gas is provided by a power unit.

11. The fast glue replenishment method by using the dispensing cartridge of claim 9, wherein in the step of regulating the gas to have the gas pressure value, the gas is regulated to have the gas pressure value by a regulation unit.

12. The fast glue replenishment method by using the dispensing cartridge of claim 9, wherein in the step of storing the gas having the gas pressure value, the gas having the gas pressure value is stored in a gas container.

13. The fast glue replenishment method by using the dispensing cartridge of claim 9, wherein in the step of controlling the gas having the gas pressure value to be output, the gas having the gas pressure value is controlled to be output by a switch of a control unit.

14. The fast glue replenishment method by using the dispensing cartridge of claim 13, wherein the control unit utilizes the switch to control the gas in the gas container enters the dispensing cartridge or not.

15. The fast glue replenishment method by using the dispensing cartridge of claim 13, further comprising a step of timing controlling, and in the step of timing controlling, an on/off period and/or on/off times of the switch is controlled by a timing controller.

16. The fast glue replenishment method by using the dispensing cartridge of claim 13, wherein in the step of outputting the gas having the gas pressure value into the dispensing cartridge, the dispensing cartridge has an accommodating space communicative to an air inlet and a feed port, and the air inlet is communicative to the control unit.

17. The fast glue replenishment method by using the dispensing cartridge of claim 16, further comprising a step of monitoring the gas pressure, in the step of monitoring the gas pressure, the gas pressure of the gas in the accommodating space is monitored by a gas pressure monitor.

18. The fast glue replenishment method by using the dispensing cartridge of claim 16, wherein the dispensing cartridge further comprises a push rubber piston part, and the push rubber piston part is slidably disposed in the accommodating space.
